(19) United States
(12) Patent Application Publication
(10) Pub. No.: US 2015/0290323 A1
(43) Pub. Date: Oct. 15, 2015

(54) DIALKYL TRISULFIDES AND FORMULATIONS OF DIALKYL TRISULFIDES FOR USE AS A CYANIDE ANTIDOTE

(71) Applicant: Sam Houston State University, Huntsville, TX (US)

(72) Inventors: Ilona Petrikovics, Huntsville, TX (US); Kristof I. Kovacs, Huntsville, TX (US)

(21) Appl. No.: 14/685,028

(22) Filed: Apr. 13, 2015

Related U.S. Application Data
(60) Provisional application No. 61/978,514, filed on Apr. 11, 2014.

(51) Int. Cl. A61K 47/24; A61K 47/40; A61K 47/10; A61K 33/00; A61K 9/107; A61K 45/06; A61K 33/04; A61K 31/105; A61K 47/44

(52) U.S. Cl. CPC A61K 47/24; A61K 31/105; A61K 47/40; A61K 47/10; A61K 47/44; A61K 9/107; A61K 45/06; A61K 33/04; A61K 33/00

(57) ABSTRACT

Dialkyl trisulfide antidote compositions may be used to as a cyanide poisoning antidote. Formulations of dialkyl trisulfide may be made in an aqueous solvent system that includes water, a co-solvent and/or a surfactant.

### PRIORITY CLAIM

This application claims priority to U.S. Provisional Application Ser. No. 61/978,514 entitled “DIALKYLTRISULFIDES AND FORMULATIONS OF DIALKYLTRISULFIDES FOR USE AS A CYANIDE ANTIDOTE” filed Apr. 11, 2015, which is incorporated herein by reference in its entirety.

### BACKGROUND

1. Field of the Invention

The present invention relates to antidotes for blood agents. More particularly, the invention relates to cyanide antidotes.

2. Brief Description of the Related Art

Cyanide (CN) intoxication in humans can occur in a number of scenarios including as part of a chemical weapon-based military conflict. CN causes rapid and extensive cellular hypoxia through the binding of the ferric (Fe³⁺) iron in the cytochrome c oxidase system leading to the collapse of the electron transport chain and thereby inhibiting the efficiency of oxygen transport to the tissues. Common cyanide compounds include hydrogen cyanide gas, cyanogen chloride gas, and crystalline solids such as potassium cyanide and sodium cyanide. The ease of delivery of these agents (especially gaseous cyanides) allow them to been used as an attack agent in chemical warfare.

Therapeutic attempts to counteract cyanide poisoning have been developed to inhibit the toxic effects of cyanide. For example, oxygen, sodium thiosulfate, amyl nitrite, sodium nitrite, 4-dimethylaminophenol, hydroxocobalamin, dicobalt EDTA, garlic extracts, disulfides, sodium pyruvate, alpha-keto-glutaric acid, aqueous solutions of ferrous sulfate in a citric acid sodium carbonate solution have been for cyanide detoxification.

Presently in the United States two kits have been accepted as the standard of care. One is based on the intravenous administration of a combination of sodium nitrite (SN) and sodium thiosulfate (TS) (Nithiodote®), while the other intravenously used preparation contains hydroxocobolamin (Cyanokit®). Hydroxocobalamin binds to CN and forms cyanocobalamin, which is then excreted in the urine. Sodium nitrite leads to the formation of methemoglobin which has high affinity to CN and forms a relative stable complex of cyanomethemoglobin. Recent studies have focused on the nitric oxide formation by nitrites, and the modulator effects of nitric oxide on Cytochrome c Oxidase inhibition. Acting as a sulfur donor, TS helps bolster the natural CN detoxification by endogenous sulfur transferases, such as rhodanese (Rh), which utilize sulfur and convert CN into thiocyanate.

U.S. Pat. No. 4,565,311 to Sarnoff, which is incorporated herein by reference, describes as an antidote for cyanide poisoning injectable hydroxylamine hydrochloride. This is followed by treatment with thiosulfate. The hydroxylamine hydrochloride can also be employed as a respiratory stimulant in treating other illnesses.

Zottola et al. in “Disulfides as Cyanide Antidotes: Evidence for a New In Vivo Oxidative Pathway for Cyanide Detoxification.” Chemical Research Toxicology, 2009, 22, pp. 1948-1953, which is incorporated herein by reference, describes the conversion of cyanide to thiocyanate in the presence of the enzyme rhodanese. Rhodanese is an enzyme found primarily in the mitochondria. In a mammal, rhodanese is thought to be responsible for the conversion of cyanide to thiocyanate (SCN). Thiocyanate is then excreted by the kidney. Oxidized sulfur species such as sodium thiosulfate have been shown to be effective in vitro donors for rhodanese, however sodium thiosulfate in vivo efficacy is highly limited due to its limited cell penetration capability to reach the endogenous rhodanese. Thus, more effective sulfur analogs are desired.

The present therapies of sodium thiosulfate (TS) and sodium nitrite (SN) (Nithiodote), and the hydroxocobalamin (Cyanokit) both have limitations of requiring intravenous administration. Additionally, TS is highly dependent on the presence of sulfurtransferase enzyme (Rhodanese), and cannot easily penetrate through the mitochondrial membrane to reach the endogenous Rhodanese. The Cyanokit requires high volume of administration to reach the required dose. There is, therefore, a need to develop a new, fast acting cyanide antidote, that can be administered in a way that provides rapid absorption to protect individuals without requiring specialized techniques such as intravenous injection.

### SUMMARY

Cyanide antidote methods are described herein. In some embodiments, a method of treating cyanide intoxication in a subject, comprises administrating to a subject who would benefit from such treatment a therapeutically effective amount of dialkyl trisulfides (DATS). The DATS may be administered as a solution intramuscularly.

A pharmaceutical composition for treating cyanide intoxication in a subject, includes a dialkyl trisulfide formulated in a solvent system, such as co-solvent and/or a surfactant. These formulation methods are used to make the lipid soluble dialkyl trisulfide water soluble, making it appropriate for intramuscular administration. In another embodiment, lipid based micelles may also be applicable for intramuscular administration.

In an embodiment, the solvent system may be composed of water and a co-solvent. The co-solvent may be an alcohol (e.g., ethanol, polyethylene glycol, etc.) or an ether (e.g., polyethylene glycol (PEG)).

In an embodiment, the solvent system may be composed of water and a surfactant. The surfactant may be a non-ionic surfactant (e.g., ethoxylated castor oil). In some embodiments, a cyclodextrin may be used to improve the water solubility of the DATS.

In an embodiment, the solvent system comprises water, a surfactant, and a co-solvent.

In an embodiment, a method of treating cyanide intoxication in a subject, includes administrating to a subject who would benefit from such treatment a therapeutically effective amount of a pharmaceutical composition comprising a dialkyl trisulfide solubilized in an aqueous solvent system, wherein the aqueous solvent system comprises: water, a co-solvent and/or a surfactant. The dialkyl trisulfide pharmaceutical composition may be administered intramuscularly, intraosseously, using an aerosol delivery system or via other commonly accepted means of administration.

### BRIEF DESCRIPTION OF THE DRAWINGS

Features and advantages of the methods and apparatus of the present invention will be more fully appreciated by reference to the following detailed description of presently preferred but nonetheless illustrative embodiments in accordance with the present invention when taken in conjunction with the accompanying drawings in which:

FIG. 1 shows the CN to SCN conversion rate of MPTS and TS;

FIG. 2 shows the solubility of MPTS in various water/co-solvent systems;

FIG. 3 shows the solubility of MPTS in the various water/surfactant solvent systems;

FIG. 4 shows the solubility of MPTS in various solvent systems that include water, a co-solvent, and a surfactant; and

FIG. 5 depicts the solubility of MPTS in a solvent system that includes water and various concentrations of Cremophor EL and ethanol.

While the invention is susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents and alternatives falling within the spirit and scope of the present invention as defined by the appended claims.

### DETAILED DESCRIPTION

It is to be understood that the present embodiments are not limited to particular compounds, methods or biological systems, which may, of course, vary. It is also to be understood that, as used in this specification and the appended claims, the singular forms “a”, “an”, and “the” include singular and plural referents unless the content clearly dictates otherwise. It is to be yet further understood that any terminology used herein is for the purpose of describing particular embodiments only, and is not intended to be limiting.

The terms used throughout this specification generally have their ordinary meanings in the art, within the context of the invention, and in the specific context where each term is used. Certain terms are discussed below or elsewhere in the specification, to provide additional guidance to the practitioner in describing the general embodiments of the invention, as well as how to make and use them. It will be readily appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

As used herein, phrases such as “one or more additional compositions or medicaments suitable for the treatment of the toxic effects of cyanide in a subject,” or more simply, “one or more additional compositions or medicaments,” generally refer to a pharmaceutical composition that contains at least one pharmaceutically active compound that is used for the treatment of the toxic effects of cyanide in a subject, but which is distinct from the sulfur analogs or derivatives that form the basis of the present disclosure.

As used herein “cyanide intoxication” is to be understood to mean a medical condition that is characterized by cyanide interference with the performance of the cytochrome oxidase system thereby inhibiting the efficiency of oxygen transport to the tissues.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein the terms “administration,” “administering,” or the like, when used in the context of providing a pharmaceutical composition to a subject generally refers to providing to the subject one or more pharmaceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or intoxicated state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

As used herein the terms “subject” generally refers to a mammal, and in particular to a human.

The terms “in need of treatment,” “in need thereof,” “who would benefit from such treatment,” or the like when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal has been exposed to cyanide and that may be detoxified, ameliorated, or treated with the specified medical intervention.

The phrases “therapeutically effective amount” and “effective amount” are synonymous unless otherwise indicated, and mean an amount of a compound of the present invention that is sufficient to improve the condition, disease, or disorder being treated. Determination of a therapeutically effective amount, as well as other factors related to effective administration of a compound of the present invention to a patient in need of treatment, including dosage forms, routes of administration, and frequency of dosing, may depend upon the particulars of the condition that is encountered, including the patient and condition being treated, the severity of the condition in a particular patient, the particular compound being employed, the particular route of administration being employed, the frequency of dosing, and the particular formulation being employed. Determination of a therapeutically effective treatment regimen for a patient is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the mammalian host treated and the particular mode of administration.

The term “pharmacologically inert,” as used herein, generally refers to a compound, additive, binder, vehicle, and the like, that is substantially free of any pharmacologic or “drug-like” activity.

A “pharmaceutically acceptable formulation,” as used herein, generally refers to a non-toxic formulation containing a predetermined dosage of a pharmaceutical composition, wherein the dosage of the pharmaceutical composition is adequate to achieve a desired biological outcome. The meaning of the term may generally include an appropriate delivery vehicle that is suitable for properly delivering the pharmaceutical composition in order to achieve the desired biological outcome. By way of non-limiting example, a composition may be administered parenteral, subcutaneous, intravenous, intracoronary, rectal, intramuscular, intra-peritoneal, intraosseous, transdermal, or buccal routes of delivery. Alternatively, or concurrently, administration may be by the oral route. The dosage administered will be dependent upon the age, health, weight, and/or intoxicated state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

A method of treating cyanide intoxication in a subject includes administering to a subject who would benefit from such treatment a therapeutically effective amount of a dialkyl trisulfide. The amount administered is to convert at least some cyanide ions in the subject to SCN. The SCN may be excreted by the subject's kidney system.

In an embodiment, a dialkyl trisulfide has the structure:

R¹—S—S—S—R²

wherein R¹ is a C₂-C₂₀ alkyl and R² is C₁-C₂₀ alkyl. Specific examples of dialkyl trisulfides include, but are not limited to: methyl ethyl trisulfide; methyl propyl trisulfide; methyl n-butyl trisulfide; diethyl trisulfide; ethyl propyl trisulfide; ethyl n-butyl trisulfide; dipropyl trisulfide, propyl n-butyl trisulfide, and di(n-butyl)trisulfide.

Dialkyl trisulfides exhibit inadequate aqueous solubility making the formulation of liquid dosage forms of dialkyl trisulfides difficult. In some embodiments, the low aqueous solubility of dialkyl trisulfides may be resolved by dissolving dialkyl trisulfides in a pharmaceutically acceptable solvent system or carrier. In the case of dialkyl trisulfides, specifically it is desirable to utilize a solvent system that increases the concentration of dialkyl trisulfides. If a solvent system was not used, and water was used as the only solvent, the volume of injection to be administered, if a therapeutically active dose is provided, would generally exceed the tolerable limit. Thus a composition comprising a solvent system with a higher dialkyl trisulfide concentration would allow for a smaller injection volume which would make the use of dialkyl trisulfides for the treatment of cyanide intoxication feasible.

In one embodiment an aqueous solution having one or more dialkyl trisulfides in therapeutically effective amounts may be formed in a solvent system that includes water and an alcohol. In some embodiments, a solvent system may include water and a surfactant. In some embodiments, a solvent system may include water, an alcohol, and a surfactant.

In compositions where the therapeutically effective amount of pharmaceutically active ingredient cannot be dissolved in water alone, adequate amounts of solubilizing excipients such as co-solvents or surfactants can be used. It is well known for one skilled in the arts that the amount of organic or non-aqueous solvents or excipients, replacing water in the composition in a dosage form, especially in parenteral (intramuscular) dosage forms, is limited due to the toxicity of these excipients. It is generally accepted to apply as little non-aqueous excipients as possible.

Co-solvents are pharmaceutically acceptable excipients added to water to solubilize poorly water soluble molecules. They exert their solubility by: 1) decreasing the dielectric constant of the solvent system and 2) disrupting the secondary bonding structure of water. In an embodiment, co-solvents include, but are not limited to, alcohols and ethers. Alcohols that may be used include alkyl alcohols (e.g., methanol, ethanol, propanol, etc.) and polyols (e.g., propylene glycol, glycerol, etc.). Ethers that may be used include, but are not limited to, polyethylene glycols. Examples of polyethylene glycols that may be used in a solvent system include, but are not limited to: PEG 200; PEG 300; and PEG 400. It should be understood that the list is merely illustrative and any analog or derivate or a mixture of the stated molecules are included within the scope of the present invention.

Surfactants are pharmaceutically acceptable excipients added to water above a certain concentration, the so called critical micellar concentration to form micelles. These micelles form mainly in aqueous media and they are responsible for increasing the solubility of poorly water soluble drugs, such as dialkyl trisulfides. In one embodiment, surfactants are amphiphylic molecules belonging to the group of ionic or non-ionic surfactants. Examples of non-ionic surfactants that may be used to improve the solubility of dialkyl trisulfides include, but are not limited to polysorbates and ethoxylated castor oil (Cremophor®). It should be understood that the list is merely illustrative and any analog or derivate or a mixture of the stated molecules are included within the scope of the present invention.

Cyclodextrins may also be used to improve the solubility of dialkyl trisulfides in water. Cyclodextrins are pharmaceutically acceptable excipients added to water to increase the solubility of poorly water soluble drugs. Cyclodextrins form inclusion complexes with the poorly soluble drugs thus increasing the water solubility. In one embodiment cyclodextrins that may be used include, but not limited to, alpha-, beta- and gamma cyclodextrins and their derivatives. It should be understood that the list is merely illustrative and any analog or derivate or a mixture of the stated molecules are included within the scope of the present invention.

In an embodiment, a solvent system capable of delivering a therapeutic amount of one or more dialkyl trisulfides to a subject is composed of a mixture of water and a cyclodextrin. In an embodiment, the concentration of cyclodextrin in water may range from about 1% to about 50% by weight. Exemplary cyclodextrins that may be dissolved in water to form a solvent system include, but are not limited to: β (beta)-cyclodextrin; γ (gamma)-cyclodextrin; randomly methylated β (beta)-cyclodextrin; and hydroxypropyl β (beta)-cyclodextrin. Specific water based solvent systems that may be used to dissolve a therapeutic amount of one or more dialkyl trisulfides include, but are not limited to: 1-50% β (beta)-cyclodextrin in water; 1-50% γ (gamma)-cyclodextrin in water; 1-50% randomly methylated β (beta)-cyclodextrin in water; and 1-50% hydroxypropyl β (beta)-cyclodextrin in water.

In an embodiment, a solvent system capable of delivering a therapeutic amount of one or more dialkyl trisulfides to a subject is composed of a mixture of water and a co-solvent. The co-solvent may be an alcohol or an ether. In an embodiment, the concentration of alcohol and/or ether in water may range from about 1% to about 80%, or from 10% to 75% by weight. Exemplary alcohols and ethers that may be dissolved in water to form a solvent system include, but are not limited to: PEG 200; PEG 300; PEG 400; propylene glycol; and ethanol. Specific water based solvent systems that may be used to dissolve a therapeutic amount of one or more dialkyl trisulfides include, but are not limited to: 25-75% PEG 200 in water; 25-75% PEG 200:propylene glycol (1:1) in water; 25-75% propylene glycol in water; 25-75% PEG 300 in water; 25-75% PEG 300:propylene glycol (1:1) in water; 25-75% PEG 200:PEG 300 (1:1) in water; 25-75% propylene glycol:ethanol (1:1) in water; 25-75% PEG 200:ethanol (1:1) in water; 25-75% ethanol in water; and 25-75% PEG 300:ethanol (1:1) in water.

In an embodiment, a solvent system capable of delivering a therapeutic amount of one or more dialkyl trisulfides to a subject is composed of a mixture of water and a surfactant. In some embodiments, the solvent system is composed of water and a non-ionic surfactant. In an embodiment, the concentration of surfactant in water may range from about 1% to about 50%, or from 5% to 20% by weight. Exemplary non-ionic surfactants that may be dissolved in water to form a solvent system include, but are not limited to: polysorbates and ethoxylated castor oil (Cremophor® RH40 and Cremophor® EL). Exemplary polysorbates include, but are not limited to, Polysorbate 20, Polysorbate 40, Polysorbate 60 and Polysorbate 80. Specific water based solvent systems that may be used to dissolve a therapeutic amount of one or more dialkyl trisulfides include, but are not limited to: 1-50% Cremophor® RH 40 in water; 1-50% Cremophor® EL in water; 1-50% Polysorbate 80: Cremophor® RH 40 (1:1) in water; 1-50% Cremophor® EL:Cremophor® RH 40 in water; 1-50% Polysorbate 80: Cremophor® EL (1:1) in water; and 1-50% Polysorbate 80 in water.

In an embodiment, a solvent system capable of delivering a therapeutic amount of one or more dialkyl trisulfides to a subject is composed of a mixture of water, a surfactant, and a co-solvent. In some embodiments, the solvent system is composed of water, a non-ionic surfactant, and an alcohol and/or ether. In an embodiment, the concentration of surfactant in water may range from about 1% to about 25%, or from 5% to 20% by weight. The concentration of co-solvent may range from about 1% to about 80%, or from 10% to 75% by weight. Specific water based solvent systems that may be used to dissolve a therapeutic amount of one or more dialkyl trisulfides include, but are not limited to: 1-50% hydroxypropyl β (beta)-cyclodextrin: 10-50% PEG 400 in water; 1-50% Polysorbate 80:1-75% ethanol in water; 1-50% Cremophor® EL:1-75% ethanol in water; and 1-50% Cremophor® EL: 1-75% PEG 200 in water;

As noted above, the solvent system for the administration of one or more dialkyl trisulfides for the treatment of cyanide toxicity should be chosen to maximize the concentration of one or more dialkyl trisulfides in the solvent system, while minimizing the amount of additives used to increase the solubility of the one or more dialkyl trisulfides. In one embodiment, an optimized solvent system for the delivery of methyl-propyl trisulfide is composed of 15% and 20% Cremophor EL in water.

Micelles represent and offer an attractive avenue to developing a carrier system for the lipophilic dialkyl trisulfides molecules. Micelles are spherical structures composed of a hydrophobic core and a hydrophilic corona with sizes ranging from 5-50 nm. They may be produced by hydrating films of block co-polymers like PEG-PE. Instead of forming bilayers and subsequent liposomes, the unique structure of block co-polymers allow them to partition into a hydrophobic phase consisting of the fatty acid tails of the phospholipids surrounded by the hydrophilic groups consisting of the PEG and phosphate groups. Pegylated micelles have been proposed and used as carriers of hydrophobic anticancer drugs like paclitaxel.

In one embodiment, micelles may be formed which encapsulate one or more dialkyl trisulfides, allowing one or more dialkyl trisulfides to be dispersed in an aqueous solvent system. Micelles containing one or more dialkyl trisulfides may be formed by using a phospholipid. Phospholipids, as used herein, are natural or synthetic molecules that include a diglyceride and a phosphate containing group coupled to the diglyceride. Phospholipids that may be used to form micelles for delivery of one or more dialkyl trisulfides to subjects have the general structure:

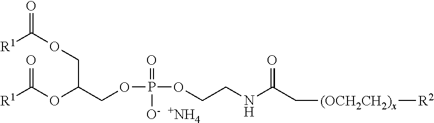

where:

R¹ is C₁₃-C₂₁ alkyl or C₁₃-C₂₁ alkenyl (monounsaturated or polyunsaturated);

X is 5-120; and

R² is hydroxyl, alkyl ether, azide, or NH₂.

Exemplary phospholipids that may be used to form micelles for the delivery of one or more dialkyl trisulfides to subjects include, but are not limited to: 1,2-dimyristoyl-sn-glycero-3-phosphoethanolamine-N-[methoxy(polyethylene glycol)-350](ammonium salt); 1,2-dipalmitoyl-sn-glycero-3-phosphoethanolamine-N-[methoxy(polyethylene glycol)-350](ammonium salt); 1,2-distearoyl-sn-glycero-3-phosphoethanolamine-N-[methoxy(polyethylene glycol)-350](ammonium salt); 1,2-dioleoyl-sn-glycero-3-phosphoethanolamine-N-[methoxy(polyethylene glycol)-350](ammonium salt); 1,2-dimyristoyl-sn-glycero-3-phosphoethanolamine-N-[methoxy(polyethylene glycol)-550](ammonium salt); 1,2-dipalmitoyl-sn-glycero-3-phosphoethanolamine-N-[methoxy(polyethylene glycol)-550](ammonium salt); 1,2-distearoyl-sn-glycero-3-phosphoethanolamine-N-[methoxy(polyethylene glycol)-550](ammonium salt); 1,2-dioleoyl-sn-glycero-3-phosphoethanolamine-N-[methoxy(polyethylene glycol)-550](ammonium salt); 1,2-dimyristoyl-sn-glycero-3-phosphoethanolamine-N-[methoxy(polyethylene glycol)-750](ammonium salt); 1,2-dipalmitoyl-sn-glycero-3-phosphoethanolamine-N-[methoxy(polyethylene glycol)-750](ammonium salt); 1,2-distearoyl-sn-glycero-3-phosphoethanolamine-N-[methoxy(polyethylene glycol)-750](ammonium salt); 1,2-dioleoyl-sn-glycero-3-phosphoethanolamine-N-[methoxy(polyethylene glycol)-750](ammonium salt); 1,2-dimyristoyl-sn-glycero-3-phosphoethanolamine-N-[methoxy(polyethylene glycol)-1000](ammonium salt); 1,2-dipalmitoyl-sn-glycero-3-phosphoethanolamine-N-[methoxy(polyethylene glycol)-1000](ammonium salt); 1,2-distearoyl-sn-glycero-3-phosphoethanolamine-N-[methoxy(polyethylene glycol)-1000](ammonium salt); 1,2-dioleoyl-sn-glycero-3-phosphoethanolamine-N-[methoxy(polyethylene glycol)-1000](ammonium salt); 1,2-dimyristoyl-sn-glycero-3-phosphoethanolamine-N-[methoxy(polyethylene glycol)-2000](ammonium salt); 1,2-dipalmitoyl-sn-glycero-3-phosphoethanolamine-N-[methoxy(polyethylene glycol)-2000](ammonium salt); 1,2-distearoyl-sn-glycero-3-phosphoethanolamine-N-[methoxy(polyethylene glycol)-2000](ammonium salt); 1,2-dioleoyl-sn-glycero-3-phosphoethanolamine-N-[methoxy(polyethylene glycol)-2000](ammonium salt); 1,2-dimyristoyl-sn-glycero-3-phosphoethanolamine-N-[methoxy(polyethylene glycol)-3000](ammonium salt); 1,2-dipalmitoyl-sn-glycero-3-phosphoethanolamine-N-[methoxy(polyethylene glycol)-3000](ammonium salt); 1,2-distearoyl-sn-glycero-3-phosphoethanolamine-N-[methoxy(polyethylene glycol)-3000](ammonium salt); 1,2-dioleoyl-sn-glycero-3-phosphoethanolamine-N-[methoxy(polyethylene glycol)-3000](ammonium salt); 1,2-dimyristoyl-sn-glycero-3-phosphoethanolamine-N-[methoxy(polyethylene glycol)-5000](ammonium salt); 1,2-dipalmitoyl-sn-glycero-3-phosphoethanolamine-N-[methoxy(polyethylene glycol)-5000](ammonium salt); 1,2-distearoyl-sn-glycero-3-phosphoethanolamine-N-[methoxy(polyethylene glycol)-5000](ammonium salt); 1,2-dioleoyl-sn-glycero-3-phosphoethanolamine-N-[methoxy(polyethylene glycol)-5000](ammonium salt); 1,2-distearoyl-sn-glycero-3-phosphoethanolamine-N-[azido(polyethylene glycol)-2000](ammonium salt); 1,2-distearoyl-sn-glycero-3-phosphoethanolamine-N-[succinyl(polyethylene glycol)-2000](ammonium salt); 1,2-distearoyl-sn-glycero-3-phosphoethanolamine-N-[carboxy(polyethylene glycol)-2000](ammonium salt); 1,2-distearoyl-sn-glycero-3-phosphoethanolamine-N-[maleimide(polyethylene glycol)-2000](ammonium salt); 1,2-distearoyl-sn-glycero-3-phosphoethanolamine-N-[PDP(polyethylene glycol)-2000](ammonium salt); 1,2-distearoyl-sn-glycero-3-phosphoethanolamine-N-[amino(polyethylene glycol)-2000](ammonium salt); 1,2-distearoyl-sn-glycero-3-phosphoethanolamine-N-[biotinyl(polyethylene glycol)-2000](ammonium salt); 1,2-distearoyl-sn-glycero-3-phosphoethanolamine-N-[cyanur(polyethylene glycol)-2000](ammonium salt); 1,2-distearoyl-sn-glycero-3-phosphoethanolamine-N-[folate(polyethylene glycol)-2000](ammonium salt); and 1,2-distearoyl-sn-glycero-3-phosphoethanolamine-N-[folate(polyethylene glycol)-5000](ammonium salt).

Micelles may be formed by the optimized process set forth below. The phospholipid, in most instances may be commercially obtained in powder form or as a solution in a suitable solvent (e.g., chloroform). In the case of the phospholipid being in powder form, the phospholipid is dissolved/suspended in ethanol. If obtained as a solution in chloroform, the chloroform The ethanol is then removed (e.g., by evaporation) to leave a liquid film. The liquid film is hydrated with water to form phospholipid micelles. Excess dialkyl trisulfide is added to the hydrated phospholipid composition. The mixture of one or more dialkyl trisulfides and micelles was subjected to ultrasound or vortex mixing for a time sufficient to create a composition of micelles of one or more of the dialkyl trisulfides dispersed in water.

One or more of the additional compounds suitable for the treatment of the cyanide intoxication presently contemplated may be formulated as a separate pharmaceutical composition to be administered in conjunction with the subject sulfur analogs as part of a therapeutic regimen, or may be formulated in a single preparation together with the sulfur analog. A combined composition may be administered orally, parenterally, by inhalation spray, rectally, or topically in dosage unit formulations containing conventional nontoxic pharmaceutically acceptable carriers, adjuvants, and vehicles as desired. Topical administration may involve the use of transdermal administration such as transdermal patches or iontophoresis devices. The term parenteral generally embraces non-oral routes of administration, including but not limited to, subcutaneous injections, intravenous, intramuscular, intrasternal injection, or infusion techniques.

Cyanide poisoning can cause death quickly in the victims that ingest, inhale, or even come into contact with substances that produce systemic cyanide poisoning in the victim. Death from cyanide poisoning can occur in less than 24 hours, generally 2-6 hours depending how the cyanide was administered, and the amount of cyanide the victim was exposed to. For the treatment of cyanide poisoning it is therefore important to be able to administer an effective detoxification agent quickly. Most commercially available cyanide poisoning treatments are designed for intravenous injection. While intravenous injection allows rapid delivery of the detoxification agent, it requires a skilled person to administer properly. It is important to have an administration method that is suitable for a large untrained population. Intramuscular or subcutaneous administration would achieve this goal, since the injection site would not be critical. In some embodiments, the injection may be administered into the muscle of the patient (i.e., intramuscular injection). In another embodiment, one or more dialkyl trisulfides may be administered by subcutaneous injection.

Therapeutic kits that include one or more dialkyl trisulfides are also contemplated herein. Such kits will generally contain, in suitable container, a pharmaceutically acceptable formulation of the dialkyl trisulfide. The kits also may contain other pharmaceutically acceptable formulations, such as those containing components to target the dialkyl trisulfide to distinct regions of a patient where treatment is needed as well as appropriate devices for delivery of the dimethyl sulfide to the subject (e.g., an injection device).

The kits may have a single container that contains the one or more dialkyl trisulfides, with or without any additional compositions or medicaments, or they may have distinct container means for each desired composition. The container of the kit will generally include at least one vial, test tube, flask, bottle, syringe or other container, into which the one or more dialkyl trisulfides, and any other desired agent, may be placed and, preferably, suitably aliquoted. Where additional components are included, the kit will also generally contain a second vial or other container into which these are placed, enabling the administration of separated designed doses. The kits also may comprise a second/third container means for containing a sterile, pharmaceutically acceptable buffer or other diluent/excipient.

The kits also may contain a device to administer the pharmaceutical compositions to an animal or patient, e.g., one or more needles or syringes, or even an eye dropper, pipette, or other such like apparatus, from which the formulation may be injected into the animal. The kits may also include a means for containing the vials, or such like, and other component, in close confinement for commercial sale, such as, e.g., injection or blow-molded plastic containers into which the desired vials and other apparatus are placed and retained.

Non-limiting examples of dialkyl trisulfide efficacy and formulation testing are described herein.

### Examples

### Materials and Methods

### Materials

Materials for the conversion tests were potassium cyanide (KCN), formaldehyde, ferric nitrate reagent, monobasic sodium phosphate monohydrate and dibasic sodium phosphate anhydrous (VWR International, Suwanee, Ga., USA). Methyl propyl trisulfide (MPTS, 50% purity; water solubility=0.15±0.003 mg/ml) was purchased from Sigma-Aldrich (St. Louis, Mo., USA), TS were purchased from VWR International (Suwanee, Ga., USA). Ethanol, PEG 200, PEG 300, PEG 400, PG (VWR International, Suwanee, Ga., USA), Cremophor EL, Cremophor RH 40, sodium cholate, sodium deoxycholate, polysorbate 80 (Sigma Aldrich, St. Louis, Mo., USA) were used as solubilizers. Cyclohexanone (Sigma Aldrich, St. Louis, Mo., USA) was used as solvent for the GC-MS measurements. KCN solutions (1.0 mg/ml and 3.5 mg/ml) were used throughout the animal studies. 250, 100 and 50 μL Hamilton Luer-lock syringes (VWR International, Suwanee, Ga., USA) were used in the animal studies with 27G 1/2 needles for intramuscular and 25G 1½ needles (VWR International, Suwanee, Ga., USA) for subcutaneous injection.

### In Vitro Efficacy Test

In vitro efficacy of MPTS was determined based on its ability to convert CN to SCN. The method applied was a spectrophotometric measurement of the formed SCN based on the method of Westley (Westley, J., 1981. Thiosulfate:Cyanide sulfurtransferase (rhodanese). Meth. Enzymol. 77, 285-291.) with minor modifications (Petrikovics, I., Cannon, E. P., McGuinn, W. D., Pei, L., Way, J. L., 1995. Cyanide antagonism with organic thiosulfonates and carrier red blood cells containing rhodanese. Fund. Appl. Toxicol. 24, 1-8.). Briefly, 200 μl of various concentrations of SDs, 200 μl of 10 mM phosphate buffered saline, 200 μl of 250 mM KCN and 400 μl of deionized water were mixed. The reaction was incubated for five minutes and was quenched with 500 μl of 15% (v/v) formaldehyde. 1.5 ml of ferric nitrate reagent was added to form a reddish brown complex (Fe(SCN)₃) that was quantitatively determined at 464 nm using a spectrophotometer (Thermo Fisher Scientific, Waltham, Mass., USA). Tests were performed with MPTS and TS at concentrations ranging from 25 mM to 0.156 mM with two fold serial dilutions in between.

### Solubility Studies

The solubility of MPTS was determined in co-solvents, surfactants and their combinations. Aqueous solutions of co-solvents and surfactants were prepared at 10%, 25%, 50%, 75%, 90% and 1%, 5%, 10%, 15%, 20% respectively. Based on the solubility enhancing efficacy of the co-solvent/water and surfactant/water systems the most effective excipients were combined into one system forming a co-solvent/surfactant/water system. Thus combinations of Cremophor EL, ethanol and PEG200 were prepared, where the concentration of the surfactant was 5%, 10%, 15%, 20% and the concentration of the co-solvents was 50%, 62.5% and 75%. Triplicates of the solvent systems were prepared in glass vials, excess MPTS was added to the solutions and the vials were sealed to eliminate the possibility of evaporation. The samples were then vortexed (Heidolph Multi Reax, Heidolph Instruments, and Cinnaminson, N.J., USA) for 20 minutes and left to equilibrate at room temperature. After equilibration (determined as 1 week) an aliquot of the samples was centrifuged (Galaxy 20R, VWR International, Suwanee, Ga., USA) at 5000 rpm for 5 minutes to ensure sedimentation of the excess MPTS and the drug content of the saturated solution was measured using a GC-MS method. Prior to GC-MS measurements the internal standard (1 mg/ml of dibuthyl disulfide; DBDS) was added to the samples and dilution with ethanol and cylcohexanone was performed.

### GC-MS Measurement

A GC-MS method was chosen for the quantitative determination of MPTS. The system consisting of an Agilent Technologies 7890A GC with a 7683 autosampler and a 5975C VL MSD, triple-Axis detector (Agilent Technologies, Santa Clara, Calif., USA). A DB-5MS column (30 m×0.25 mm ID, 0.25 ™ film thickness; Agilent Technologies, Santa Clara, Calif., USA) was used with He carrier gas at a flow rate of 1 ml/min and pressure of 7.6522 psi. The conditions for GC and MS are detailed in Tables 1 and 2.

TABLE 1
Gas chromatograph parameters

| Injection Source: | GC Auto-loading sampler (ALS) | |
|---|---|---|
| Injection Volume: | 1.0 | μL |
| Injection Port Temperature: | 250° | C. |
| Injection Mode: | Split | |
| Split Ratio: | 60:1 | |
| Carrier Gas: | helium | |
| Carrier Gas Velocity: | 1.0 | ml/min |
| Carrier Gas Pressure: | 7.6522 | psi |
| Initial Temperature of Column: | 50° | C. |
| Initial Temperature Duration: | 2 | mins |
| Temperature Ramp: | 5° | C./min |
| Final Temperature of Column: | 250° | C. |
| Final Temperature Duration: | 5 | mins |

TABLE 2
Mass spectrometer parameters

| EMV Mode: | Relative (+200) | |
|---|---|---|
| EM Voltage: | 1118 | |
| Solvent Delay: | 2.00 | mins |
| Source temperature: | 230° | C. |
| Quadrupole temperature: | 150° | C. |
| Electron energy: | 70 | eV |

LD50 studies were conducted using the Dixon up-and-down method with 1.0 mg/ml and 3.5 mg/ml KCN solutions, a 50 mg/ml MPTS stock solution, and a 100 mg/ml TS solution. Male CD-1 mice (Charles River Breeding Laboratories, Inc., Wilmington, Mass.) weighing 18-28 g were housed at 21° ° C. and in light-controlled rooms (12-h light/dark, full-spectrum lighting cycle with no twilight), and were furnished with water and 4% Rodent Chow (Teklad HSD, Inc., CITY, Wis.) ad libitum. All animal procedures were conducted in accordance with the guidelines by “The Guide for the Care and Use of Laboratory Animals” (National Academic Press, 2010), accredited by AAALAC (American Association for the Assessment and Accreditation of Laboratory Animal Care, International). At the termination of the experiments, surviving animals were euthanized in accordance with the 1986 report of the AVMA Panel of Euthansia.

Animal studies were conducted as therapeutic experiments using the up-and-down method for LD50 determination and the estimated 95% confidence interval was also calculated (Dixon, W. J., 1965. The up-and-down method for small animal samples. Am. Stat. Assoc. J. 12, 967-978). Based on the weight of the animal an initial dose of KCN was injected subcutaneously from the KCN stock solution. Within 30 seconds, based on the weight of the animal, a predetermined dose (either 100 mg/kg or 200 mg/kg) of MPTS (50 mg/ml in 10% Cremophor EL+50% ethanol) or TS (100 mg/ml in water) was injected intramuscularly into the rear right leg of the mouse. In case of the combination studies MPTS was injected intramuscularly into the right leg, TS intramuscularly into the left leg both within 30 seconds of the KCN administration. The mice were then inspected and determined to be alive or dead. Based on the observation, a higher or a lower dose of KCN was injected in the following stage. This was repeated until enough data was collected to determine the LD50 values, and the computer declared that the stopping condition has been met. For each LD50 determination, 9-14 animals were used.

### Results and Discussion

### In Vitro Efficacy Test

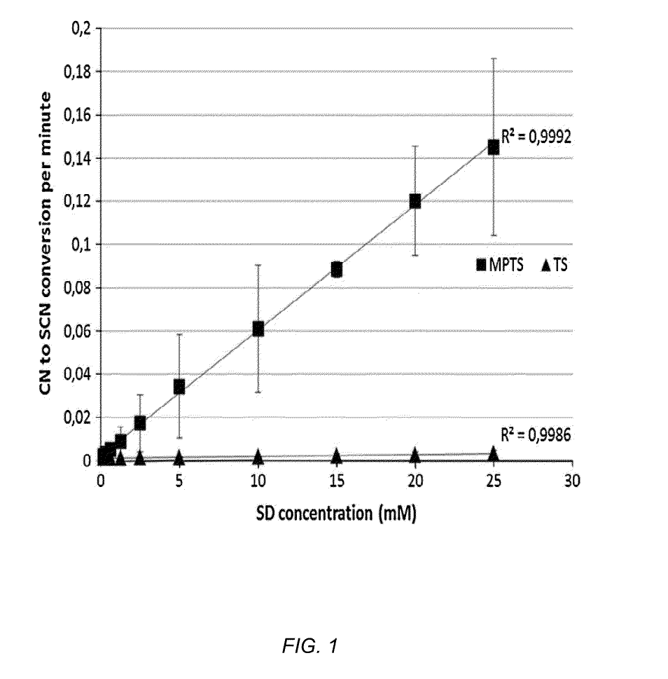

In the first set of experiments the in vitro efficacy of MPTS was tested in order to determine its efficiency in converting CN to SCN. This effect was then compared to that of TS, which is used as the SD component in one of the currently approved CN antidote kits. Comparison of its activity with that of MPTS would thus give a valuable insight on the in vitro efficacy of MPTS. FIG. 1 shows the CN to SCN conversion rate of MPTS and TS.

The results show that the conversion rate produced by MPTS is higher than that of TS at all tested concentrations, indicating the usefulness of the newly tested molecule in combating CN intoxication. A 2 fold increase in conversion rate was already seen at concentrations as low as 0.156 mM and as the concentration of the two SDs increased the relative efficacy of MPTS compared to TS increased to a substantial 44 fold at 25 mM SD concentration. It was also seen that the reaction rates are directly proportional to the concentrations of MPTS and TS (equation MPTS: y=0.0058x+0.0024; R²=0.9992; equation TS: y=0.00008x+0.0011; R²=0.9986) indicating that the efficacy of MPTS in future in vivo studies might prove to be dose dependent. Based on these in vitro findings it can be concluded that MPTS is an effective sulfur donor and therefore solubilization of the drug for intramuscular in vivo studies was initiated.

### Solubility of MPTS in Co-Solvents

Solubilization studies were divided into three steps: in the first and second steps the solubility of MPTS was determined in co-solvent/water and surfactant/water systems. In the final phase of the studies, based on the results of the first two stages, the most effective surfactant and co-solvents were combined into one system and the solubility of the antidote candidate molecule was determined in such systems in the hope of further increasing its solubility.

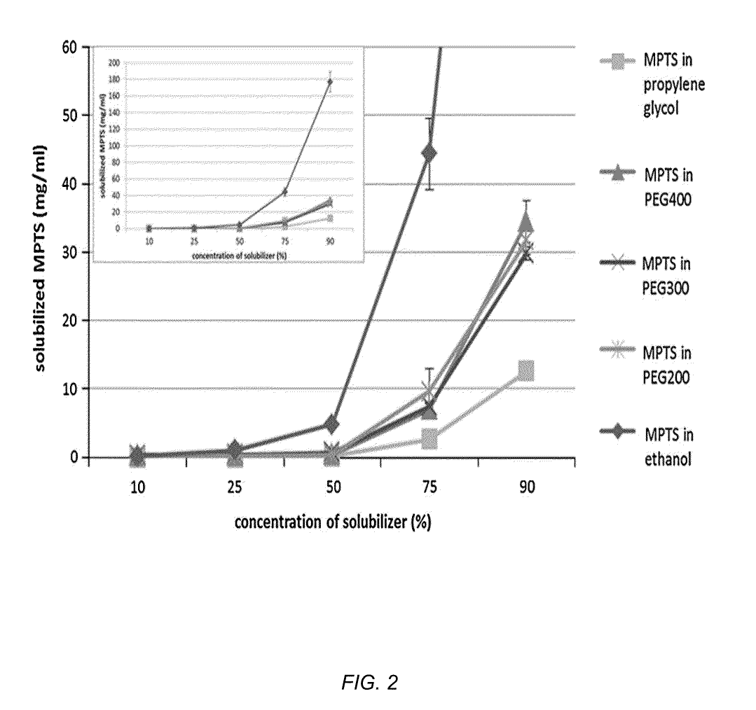

The effect of co-solvent/water systems on the solubility of MPTS was examined using ethanol, PEG 200, PEG 300, PEG 400 and propylene glycol at concentrations of 10%, 25%, 50%, 75% and 90%. FIG. 2 shows the solubility of MPTS in these co-solvent/water systems. The inserted figure shows the solubilized drug concentrations up to a higher value, while the large figure shows the values up to a lower concentration so as to facilitate the distinction between the solubilizing effects of the PEGs.

The solubility enhancing effect attributed to the co-solvents can be explained a) by their ability to interrupt the hydrogen bonding structure of the water molecules, thus decreasing the squeezing out effect of non-polar molecules from the polar solvent; and b) by their ability to decrease the dielectric constant of the solvent system. The exponential solubility curve seen in the case of MPTS (FIG. 2) correlates well with the previously published solubility tests using co-solvents. These studies reported that a linear increase in the concentration of the co-solvent increases the solubility of drugs exponentially. Results show that the most effective solubilizer is ethanol, solubilizing 177.11±12.17 mg/ml MPTS at 90% and 44.35±5.15 mg/ml MPTS at 75%. PEG200, PEG300 and PEG400 exerted similar solubility enhancing capacities, but their solubilizing power falls short of the one encountered with ethanol. Based on the solubility enhancing effect of the co-solvents, ethanol and PEG200 were picked to be included in further studies when co-solvents were combined with surfactants.

### Solubility of MPTS in Surfactants

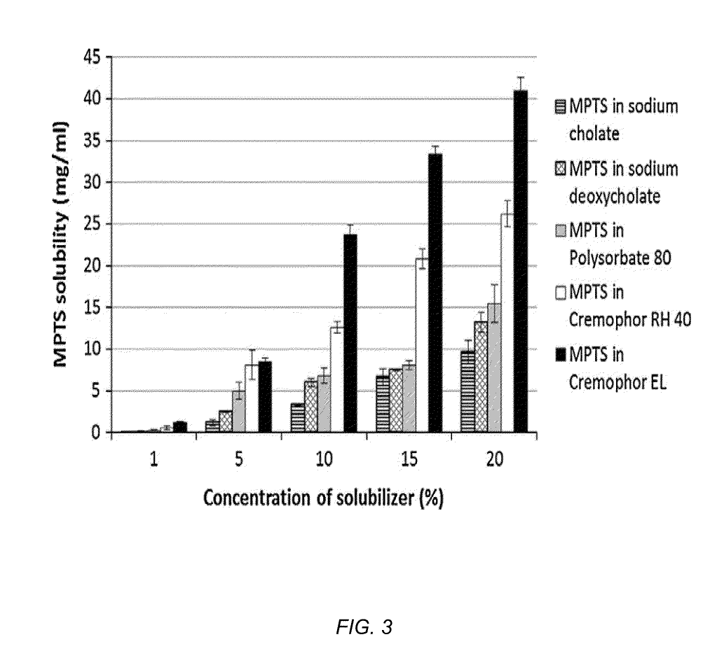

In step two of the studies, the effect of surfactant/water systems on the solubility of MPTS was examined using Cremophor EL, Cremophor RH40, polysorbate 80, sodium cholate and sodium deoxycholate at 1%, 5%, 10%, 15% and 20%. FIG. 3 shows the solubility of MPTS in the various water/surfactant solvent systems.

The solubilizing effect of surfactants rests on their ability to form micelles above the critical micellar concentration. All surfactants were used above this concentration, thus the solubilizing effect can be associated with the number and size of micelles formed. FIG. 3 shows that the solubility of MPTS increased linearly with the linear increase in the concentration of the surfactants. Out of the tested surfactants, the highest solubility of MPTS was achieved in Cremophor EL at all tested concentrations, with maximum MPTS solubility of 40.99±1.55 mg/ml at 20% Cremophor EL concentration. All the other surfactants increased the solubility of the molecule at different rates, in the following order: Cremophor EL>Cremophor RH 40>polysorbate 80>sodium deoxycholate>sodium cholate. Generally, results show that with the use of non-ionic surfactants a superior solubility enhancing effect can be achieved compared to the ionic surfactants (sodium cholate and deoxycholate). Based on the solubility of MPTS Cremophor EL was chosen for further studies.

### Solubility of MPTS in Co-Solvent Surfactant Combinations

It is well known that the amount of excipients present in a composition, especially in an intramuscular parenteral preparation, might have a significant effect on the overall toxicity of the final preparation. Therefore, it was the aim of the study to develop a composition with an adequate solubilizing power while utilizing as little amount of excipients as possible. The use of ethanol was not excluded based on the fact that the administration of a highly concentrated solution of MPTS would mean that the total volume of injection is low, therefore the administered dose of ethanol is also very low. Taking the above, and the solubility enhancing effect of co-solvents and surfactants into consideration, it was evident that a more complex system was needed to solubilize higher concentrations of the drug. Therefore, the excipients that showed the highest solubilizing power during the first two phases of the studies were combined in the hope of developing a solvent system that is capable of solubilizing higher MPTS concentrations than those seen in co-solvent/water and surfactant/water systems. Cremophor EL was chosen as the surfactant (it solubilized the most MPTS out of the surfactant type excipients), and ethanol and/or PEG200 were chosen as the co-solvents.

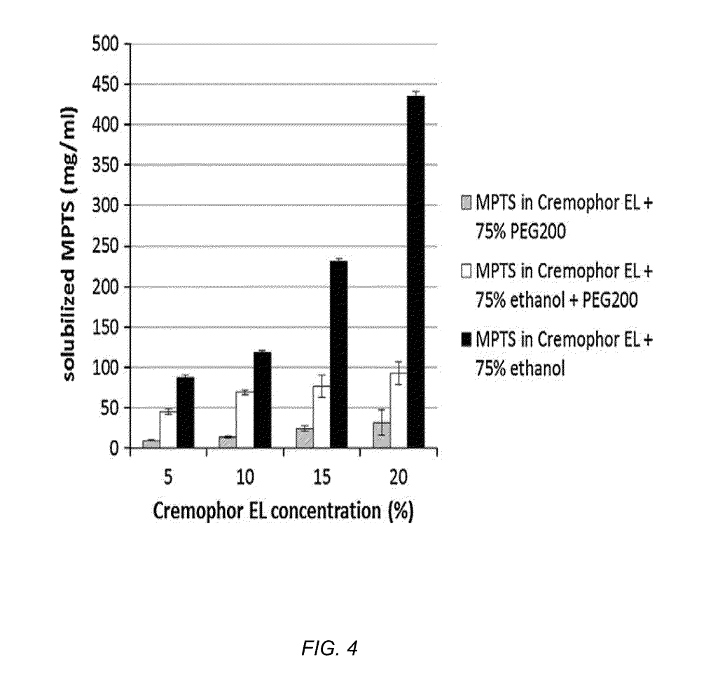

The above mentioned co-solvents were combined with increasing amounts of Cremophor EL to form the following solvent systems: surfactant+75% ethanol, surfactant+75% PEG 200, surfactant+37.5% ethanol+37.5% PEG 200 (=75% ethanol:PEG200=1:1). FIG. 4 shows the solubility of MPTS in various solvent systems that include water, a co-solvent, and a surfactant.

The solubilizing effect of the tested systems depended on the interaction of the excipients and it can be classified as negative, additive or synergistic based on how much more or less MPTS is solubilized in the surfactant/co-solvent/water combination than in the corresponding co-solvent/water and surfactant/water systems. The measured solubility of MPTS in the combination system of Cremophor EL and PEG200 was lower than the calculated solubility of the antidote candidate if the solubility values measured in Cremophor EL/water and PEG200/water were added (Table 3). In the case of the combination of Cremophor EL with ethanol and Cremophor EL with ethanol and PEG200 at a ratio of 1:1 a synergistic solubilizing effect was encountered in both systems, meaning that measuring the solubility of MPTS in the excipients separately and adding the results would lead to a lower concentration of SD than the one seen when the excipients were mixed in one system and the solubility of MPTS was determined in that solvent (Table 3). The level of synergism encountered with the two systems differed, Cremophor EL+ethanol exhibiting a larger rate.

TABLE 3
Solubility of MPTS in various systems comprising 75% co-solvent, surfactant and their combination

| | Co-Solvent | | | | | | |
|---|---|---|---|---|---|---|---|
| | MPTS solubility (mg/ml) | | | | | | |
| | | | | 75% Ethanol:PEG200 | | | |
| Cremophor EL | No co-solvent | 75% Ethanol | | (1:1) | | 75% PEG200 | |
| Conc. (%) | Measured | Calculated | Measured | Calculated | Measured | Calculated | Measured |
| 0 | — | — | 44.3 | — | 18.9 | — | 12.35 |
| 5 | 8.46 | 52.76 | 86.70 | 27.36 | 45.35 | 20.81 | 9.72 |
| 10 | 23.67 | 67.97 | 117.89 | 42.57 | 69.03 | 36.02 | 13.65 |
| 15 | 33.35 | 77.65 | 230.47 | 52.25 | 76.93 | 45.70 | 24.16 |
| 20 | 40.98 | 85.28 | 434.79 | 59.88 | 92.38 | 53.33 | 31.71 |

| | | | |
|---|---|---|---|
| Remarks | Synergistic | Synergistic | Negative |
| | solubilizing effect | solubilizing effect | solubilizing effect |

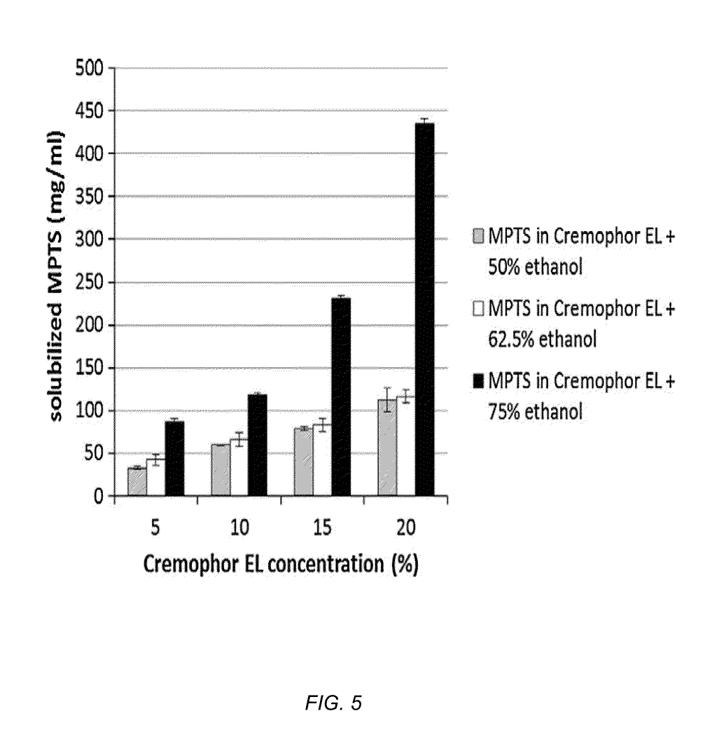

Based on the solubilizing power of the solvent systems comprising Cremophor EL in combination with ethanol or PEG200 or ethanol and PEG200 it was concluded that the combination of Cremophor EL and ethanol was the most effective solvent system for solubilizing MPTS. Furthermore, this system showed a marked synergistic solubilizing effect at 75% ethanol content. It was the aim of the research to develop a solvent system that comprises excipients in concentrations as low as possible while still exerting substantial solubilizing power, therefore, the synergistic solubilizing effect of Cremophor EL and ethanol were further studied. The solubility of MPTS was determined in Cremophor EL and ethanol combinations where the concentration of the co-solvent was decreased to 62.5% and 50%. Solubility of MPTS in such systems is presented together with the solubility values of Cremophor EL+75% ethanol (for the ease of comparison) in FIG. 5.

Results proved that the synergistic solubilizing effect encountered at 75% was also detected at 62.5% and 50% ethanol content (Table 4). As it was the aim of the experiments to reduce the concentration of the excipients as much as possible but still be able to solubilize 50 mg/ml of MPTS, the final composition that was chosen for animal studies comprised 10% Cremophor EL and 50% ethanol.

TABLE 4
Solubility of MPTS in various systems comprising 75%, 62.5% and 50% ethanol surfactant
and their combination

| | Co-Solvent | | | | | | |
|---|---|---|---|---|---|---|---|
| | MPTS solubility (mg/ml) | | | | | | |
| Cremophor EL | No co-solvent | 75% Ethanol | | 62.5% Ethanol | | 50% Ethanol | |
| Conc. (%) | Measured | Calculated | Measured | Calculated | Measured | Calculated | Measured |
| 0 | — | — | 44.3 | — | 16.3 | — | 4.8 |
| 5 | 8.46 | 52.76 | 86.70 | 24.76 | 42.37 | 13.26 | 32.75 |
| 10 | 23.67 | 67.97 | 117.89 | 39.97 | 66.51 | 28.47 | 60.14 |
| 15 | 33.35 | 77.65 | 230.47 | 49.65 | 83.01 | 38.15 | 78.71 |
| 20 | 40.98 | 85.28 | 434.79 | 57.28 | 116.32 | 45.78 | 112.69 |

| | | | |
|---|---|---|---|
| Remarks | Synergistic | Synergistic | Synergistic |
| | solubilizing effect | solubilizing effect | solubilizing effect |

### Animal Studies

As a result of the solubility studies, compositions that were able to solubilize significant amounts of MPTS were developed. A composition comprising 10% Cremophor EL, 50% ethanol and 50 mg/ml MPTS was chosen for the animal studies. The in vivo efficacy studies were performed with MPTS alone (dose=100 mg/kg and 200 mg/kg) and TS alone (dose=100 mg/kg and 200 mg/kg) and their combination with the doses of 200 mg/kg for each. Therapeutic antidotal potency ratios (APR) of the drugs and their combinations are shown in Table 5. The following were used for the calculation of the antidote potency ratio (APR) and the relative antidote potency ratio (RAPR): APR=LD50 of CN with the antidote(s)/LD50 of CN without antidote(s) (control); relative antidotal potency ratio (RAPR)=APR(1)/APR(2)

TABLE 5
Dielectric constant values of tested solvent systems

| | Average of | |
|---|---|---|
| Sample | dielectric constant | SD |
| 50% Ethanol | 53.05 | 0.0108 |
| 60% Ethanol | 47.13 | 0.0163 |
| 65% Ethanol | 44.50 | 0.0031 |
| 70% Ethanol | 41.83 | 0.0016 |
| 75% Ethanol | 38.92 | 0.0039 |
| 5% Cremophor EL + 50% ethanol | 56.42 | 0.1463 |
| 10% Cremophor EL + 50% ethanol | 50.44 | 0.0118 |
| 15% Cremophor EL + 50% ethanol | 48.66 | 0.0120 |
| 20% Cremophor EL + 50% ethanol | 45.60 | 0.0063 |

The antidotal efficacy tests (Table 6) demonstrated the superior effect of MPTS over TS (Exp. 1 vs. Exp 3; and Exp 2 vs. Exp. 4). The positive dose effects are also demonstrated: MPTS alone provided a 1.2 LD50 protection when the dose was 100 mg/kg, while the double dose (200 mg/kg) provided an enhanced protection with the APR of 1.67 (RAPR=1.39). TS alone provided only a slight protection with the APR of 1.1 when the dose was 100 mg/kg, and when the dose was doubled (200 mg/kg), the APR was enhanced to 1.25 (RAPR=1.13). Employing the same dose of 200 mg/kg for both components of the combination with MPTS and TS (Exp. 5), the antidotal protection was significantly enhanced to 3.66×LD50. The enhancement by TS was 2.19×compared to MPTS alone. The enhancement by MPTS was 2.92× compared to TS alone. The tests not only showed that MPTS is effective in combating cyanide intoxication but it also revealed that the newly identified molecule is more effective than the currently used TS. Furthermore, it was also shown that intramuscular administration is an effective way of applying the antidote as absorption of the molecule from the muscle was fast enough to counteract the toxic effects of cyanide.

TABLE 6
APR and RAPR values of the antidotes

| | | | RAPR | RAPR |
|---|---|---|---|---|
| Exp. # | Treatments | APR | (dose effect) | (MPTS vs. TS) |
| 1 | MPTS 100 mg/kg | 1.2 | | |
| | (intramuscular) | | | |
| 2 | MPTS 200 mg/kg | 1.67 | 1.67/1.2 = 1.39 | 1.67/1.25 = 1.33 |
| | (intramuscular) | | | |
| 3 | TS 100 mg/kg | 1.1 | | |
| | (intramuscular) | | | |
| 4 | TS 200 mg/kg | 1.25 | 1.25/1.1 = 1.13 | |
| | (intramuscular) | | | |
| 5 | MPTS 200 mg/kg | 3.66 | | 3.66/1.67 = 2.19 |
| | (intramuscular)+ | | | |
| | TS 200 mg/kg | | | 3.66/1.25 = 2.92 |
| | (intramuscular) | | | |

### CONCLUSION

The identification of a possible antidote for CN intoxication and the solubilization of the highly hydrophobic sulfur donor MPTS for the therapeutic antidotal studies using a lethal animal model were addressed in this study. Based on in vitro CN to SCN conversion testing of potential sulfur donors, MPTS was found to be a potentially effective molecule. The in vitro efficacy of the newly identified SD was superior to that of TS, the SD component in one of the currently approved antidote kits. Following the identification of the SD, which is a highly lipophilic molecule, its solubilization was initiated. A major issue with the compound is its very limited water solubility, thus in the first part of the solubility studies solubilizers, such as co-solvents and surfactants, were used to dissolve MPTS. These studies showed that all the applied aqueous excipient systems are effective at dissolving the SD, but a more complex system was needed to further increase the solubilizing capacity and develop a solvent that can dissolve MPTS at therapeutically relevant concentrations. Thus co-solvent-surfactant combinations were prepared and it was concluded that combinations comprising Cremophor EL and ethanol solubilize the most MPTS. Based on these studies and taking into consideration the possible toxicity of the excipients, the final preparation of 10% Cremophor EL+50% ethanol was chosen. The results of the solubility studies showed that the combination of the selected co-solvents and surfactants exerted a synergistic solubilizing effect, increasing the solubility of the antidote candidate significantly. Using the developed solvent system the antidotal efficacy of MPTS was evaluated in a lethal animal model, conducted with CD-1 male mice at two different MPTS doses (100 mg/kg and 200 mg/kg). Further combination studies were also performed with co-administration of MPTS and TS. The therapeutic antidotal potency ratio results proved the in vivo efficacy of the compound alone and in combination with TS. A very promising APR value of 3.6 was achieved with the combination of MPTS and TS. Furthermore, it was also shown that intramuscular administration is an effective way of applying the antidote as absorption of the molecule from the muscle was fast enough to counteract the toxic effects of cyanide. Based on the results, MPTS was proven to be a promising effective molecule in the fight against CN poisoning, and the proposed solvent system and administration route may serve as the base for an intramuscular parenteral dosage form of MPTS.

In this patent, certain U.S. patents and U.S. patent applications have been incorporated by reference. The text of such U.S. patents and U.S. patent applications is, however, only incorporated by reference to the extent that no conflict exists between such text and the other statements and drawings set forth herein. In the event of such conflict, then any such conflicting text in such incorporated by reference U.S. patents and U.S. patent applications is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A pharmaceutical composition for treating cyanide intoxication in a subject, comprising:
one or more dialkyl trisulfides dissolved in an aqueous solvent system, wherein the aqueous solvent system comprises: water and one or more of: a co-solvent; a surfactant; a cyclodextrin; and a phospholipid; and
wherein the one or more dialkyl trisulfides have the structure:
R¹—S—S—S—R²
wherein R¹ is a C₂-C₂₀ alkyl and R² is C₁-C₂₀ alkyl.

2. The pharmaceutical composition of claim 1, wherein one or more of the dialkyl trisulfides is methyl propyl trisulfide.

3. The pharmaceutical composition of claim 1, wherein the solvent system comprises water and a co-solvent.

4. The pharmaceutical composition of claim 2, wherein the co-solvent is an alcohol.

5. The pharmaceutical composition of claim 2, wherein the co-solvent is ethanol.

6. The pharmaceutical composition of claim 2, wherein the co-solvent is an ether.

7. The pharmaceutical composition of claim 2, wherein the co-solvent is a polyethylene glycol (PEG).

8. The pharmaceutical composition of claim 1, wherein the solvent system comprises water and a surfactant.

9. The pharmaceutical composition of claim 8, wherein the surfactant is a non-ionic surfactant.

10. The pharmaceutical composition of claim 8, wherein the surfactant is an ethoxylated castor oil.

11. The pharmaceutical composition of claim 1, wherein the solvent system comprises water and a cyclodextrin.

12. The pharmaceutical composition of claim 1, wherein the solvent system comprises water, a surfactant, and a co-solvent.

13. The pharmaceutical composition of claim 1, wherein the solvent system comprises a phospholipid capable of forming micelles, wherein the micelles comprise DMTS.

14. The pharmaceutical composition of claim 1, further comprising one or more additional compounds, wherein the additional compounds are capable of removing and/or detoxifying cyanide in a subject.

15. The pharmaceutical composition of claim 1, wherein the pharmaceutical composition further comprises sodium thiosulfate.

16. The pharmaceutical composition of claim 1, wherein the pharmaceutical composition further comprises sodium nitrite.

17. The pharmaceutical composition of claim 1, wherein the phospholipid has the structure: 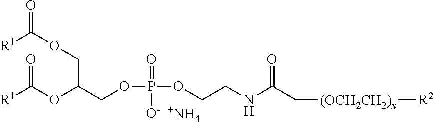
where:
R¹ is C₁₃-C₂₁ alkyl or C₁₃-C₂₁ alkenyl (monounsaturated or polyunsaturated);
X is 5-120; and
R² is hydroxyl, alkyl ether, azide, or NH₂.

18. The pharmaceutical composition of claim 1, wherein the phospholipid is 1,2-distearoyl-sn-glycero-3-phosphoethanolamine-N-[methoxy(polyethylene glycol)-2000](ammonium salt).

19. A method of treating cyanide intoxication in a subject, comprising:
administrating to a subject who would benefit from such treatment a therapeutically effective amount of a pharmaceutical composition comprising one or more dialkytrisulfides dissolved in an aqueous solvent system, wherein the aqueous solvent system comprises: water and one or more of: a co-solvent; a surfactant; a cyclodextrin; and a phospholipidand wherein the one or more dialkyl trisulfides have the structure:
R¹—S—S—S—R²
wherein R¹ is a C₂-C₂₀ alkyl and R² is C₁-C₂₀ alkyl.

20. The method of claim 19, wherein the pharmaceutical composition is administered transdermally.

21. The method of claim 19, wherein the pharmaceutical composition is administered via an aerosol delivery system.

22. The method of claim 19, wherein the pharmaceutical composition is administered as a solution subcutaneously or intramuscularly.

23-48. (canceled)
